(19) United States
(12) Patent Application Publication
(10) Pub. No.: US 2020/0139055 A1
(43) Pub. Date: May 7, 2020

(54) HOLLOW NEEDLE WITH BENDABLE HUB SYSTEM

(71) Applicant: Jasperate, Inc., Lisle, IL (US)

(72) Inventors: David Weksel, Lisle, IL (US); Stephen Soloway, Vineland, NJ (US); Charles S. Brunner, Chicago, IL (US)

(21) Appl. No.: 16/178,113

(22) Filed: Nov. 1, 2018

(51) Int. Cl. A61M 5/32

(52) U.S. Cl. CPC A61M 5/3293; A61M 25/0054

(57) ABSTRACT

A hollow needle, for use in medical and other areas where thin hollow needles are used to inject or aspirate fluids and gases, is provided and comprises of a thin, rigid tube body that is open on both ends and throughout the length with one sharp end. A flexible section is provided, either as part of the needle or as an addition at its proximal end allowing the needle to be oriented such that the needle central axis is non-colinear and non parallel to the central axis of the needling system. Depending upon the desired use, needle orientation relative to the central axis can be limited from a conical volume to a planar or multi planar angular orientations. The needling system so configured advantageously allows the user to circumvent an obstacle and or obstacles that otherwise inhibit direct linear access to a point-of-interest by providing a device that can fit around or between obstructions providing optimal direct path towards the desired therapy delivery point.

### FIELD OF THE INVENTION

The present invention concerns a needle with an integral bendable means that together form a system to ease targeting of a hard to reach point of interest with a needle. More particularly the present invention concerns a combination of a hollow needle segment and a bendable segment providing a bending means to allow the needle to avoid blockages or restrictions, obstructions, and penetrate skin, or other surfaces, so as to reach a point there within. The obstructions avoided can include those that would block a needle or that could keep a needle from reaching the point of interest by blocking a syringe or other device attached to the needle.

### BACKGROUND OF THE INVENTION

Penetration of the skin, or other surfaces, to deliver fluids, or other products, or to aspirate such from within the surface is well known. Penetration is typically done using a hollow needle in combination with any one of a variety of known devices, including but not limited to hypodermic needles. A hypodermic needle is defined as a thin tube inserted into a vein, dermis, sub dermis, muscle or fatty tissue, body cavity or the like to enable a number of different actions, including but not limited to administering medication, draining off fluid, or inserting a surgical instrument. These needles are traditionally straight, thin, hollow tubes with a sharpened tip at one end. Some variations include needles with distal openings to provide more rapid movement of fluids and other materials, extended length needles allowing deeper penetration before fluid and other material actions (such as needles used to administer adrenaline into the heart during cardiac episodes) and needles having smaller length for subcutaneous actions. Commonly used with syringes, needles provide a means to penetrate human and or other animal tissue or synthetic materials to create a pathway to target a point-of-interest. The needle and syringe combination enables the user to aspirate and or inject medication and or to draw bodily fluids such as sanguineous, serosanguineous, purulence, exudates and transudate. In most cases the needle and syringe are coaxial with each other, the syringe following the path of the needle. It is noted that in some applications a desired action using a needle can be accomplished without the use of a syringe, but instead by use of the needle alone or by integration of the needle with some other means of causing the action. A traditional hypodermic needle includes a straight, hollow needle tube and a leading cutting edge emerging directly from the attached device. Further, such needles are typically used in conjunction with a means of injecting or extracting fluids to and from a point-of-interest.

As an example, the classic combination of a needle and syringe can be considered a “needling system.” The syringe and needle are typically connected in a manner that fixes the position of the needle relative to the syringe where the needle and syringe are co-axial or in some instances the needle and syringe while not being co-axial are parallel. It is noted that the diameter of the needle is almost always significantly smaller than that of the syringe. As configured, this needling system is typically restricted to a direct line access from a surface to a point therewithin, with little variation in the direction or path. Typically, a straight needle is aimed from the surface so as to find its way, through a straight line, to a targeted position; the syringe is on that same line of access but is of significantly larger diameter than the needle. In such a case access to the target area is not only restricted by the ability of the needle to move in a straight line to the target area, but also by the ability of the syringe to bypass any potential restrictions that are exterior to the surface to be pierced.

The art of needles and needle systems for use in medical procedures has advanced little beyond the earliest uses of puncturing the skin to arrive at a straight-line position for insertion or removal of fluids. Experimental uses of hypodermic needles for medical purposes were first performed in 17th century. However, due to poor results the technology was largely dominant until the 19th century when more appropriate materials and construction techniques became available; uses also improved with the realization and implementation of sanitary conditions for medical procedures. Today, due to significant advances, hypodermic needles have emerged as a critical component of medical care worldwide.

However, a significant drawback with the traditional hollow needle and needling system, as typically configured in coaxial relationship with each other, continues to thwart their use in new and medically significant ways. Disadvantageously, in the use of traditional needles that emerge directly and coaxially from the connective device in the needling systems, there exists the problem that in the event of an obstruction either below or above the skin, the needle and/or needling system cannot easily access the point-of-interest below the skin or other tissue that requires, injection or aspiration. For example, in the medical field, a traditional straight hypodermic needle may fail when a patient has metal and or other implants embedded under the skin and or when a tendon and or other sensitive tissue blocks straight access to a joint or another point-of-interest that needs to be aspired or injected or drawn. Another example is when a patient has mobility issues or items on the outside of the skin that introduces difficulties in accessing the point-of-interest, the use of a straight needle may not give appropriate access requiring time to position the patient or remove the item blocking access to the detriment of the emergency care of the patient. Further, the structure of the body and or limits on that structure due to disease may create situations where a body part impairs access to the point of interest

Health care workers have been known to manually bend or otherwise manipulate the needle to enable access. However, the material used in the production of needles is typically not easy to manipulate and small tubes can be blocked or kinked by such manipulation such that they cannot work or will work poorly. More typically, the health care worker attempts to manipulate the needle so as to angle the straight needle around the obstruction. Unfortunately, these practices usually require using a longer and perhaps wider needle, potentially causing damage to delicate arterial, venal or other structures. Additionally, bending of the instrument by the healthcare worker puts the worker at risk of injury by having to manipulate a sharp object in an unintended/off label manner. The costs to the health care worker and patient include the added time requirements of needle preparation while issues with safety and maintaining manufacturing and structural integrity may arise. In addition, this manually bent needle can lead to inhibited flow through the needle and additional patient and health care worker trauma from the use of a larger needle. Further, the bending or other manipulation of proprietary medical devices can lead to the voiding of warranty protection on such devices and add liability to the health care worker, doctor or hospital or other institution.

It would be desirable to have a needling system that departs from a linear relationship of prior art needling systems and allows for the non-coaxial alignment of a needle and, in this non-limiting example, a syringe. In such a system the axis of the needle could be moved so that it intersects the axis of the syringe, rather than lay coaxial therewith. Further, it would be desirable in such a system, for the user to be able to decide the angle of intersection when needed and manipulate the needling system, in a manner intended by the system, and thereby use the system successfully to avoid obstacles and place the distal end of the needle at the point of interest. Such a system would save time, effort and resources as will be described in greater detail below.

It is therefore an object of the present invention to provide a device and means to access a point-of-interest in a body, where direct access to the point has been blocked by either impenetrable obstacles or obstacles that cause part or all of prior art penetration equipment to be blocked. Further, it is an object of the present invention to provide a device that can be easily manipulated to avoid such obstacles and to secure easy access to any point of interest. It is a further object of the present invention to provide a medical professional with alternative means to provide rapid and accurate health assistance in normal and emergency situations where obstructions would normally thwart the use of current state of the art hypodermic needles. Other objects and advantages of the present invention will become apparent as the description proceeds.

### SUMMARY OF THE INVENTION

In accordance with the present invention, a needling system is provided comprising a hollow needle having a distal end and a proximal end, a hub comprising means to hold the proximal end of the hollow needle at a distal end of the hub when connected thereto. The hub being receptive to connection to a supply device at a proximal end of the hub; the hub and needle sharing a longitudinal axis therethrough when connected and in a first coaxial position. Wherein the needle system is bendable, at a segment located between the needle distal end and the hub proximal end, such that the distal end of the needle can be placed at an angle relative to the longitudinal axis of the hub. It will be understood by persons having ordinary skill in the art, that the supply device can be any device that can attach to the hub to contribute to either pushing matter out through the hub and needle, or drawing matter into the needle and hub.

The present invention provides a needling system, comprising a hollow needle having a distal end and a proximal end, and a flexible section therebetween. As in most needling systems, the needle is attached, directly or indirectly, to a hub comprising means to hold the proximal end of the hollow needle at a distal end of the hub when connected thereto, and receptive to connection to, alternatively, a supply or a draw device at a proximal end of the hub, the hub and needle sharing a longitudinal axis therethrough when connected and in a first coaxial position. The needle, in the present invention is then pivotable, at its proximal end, with a flexible section, such that the distal end of the needle can be placed at an angle relative to the longitudinal axis of the hub.

In a preferred embodiment, the flexible section of the needling system comprises a plurality of circumferential grooves, each of the grooves having sides of unequal length, the flexible section being contracted lengthwise with the sides of the grooves reentrant and overlapping when in a first straight position; when the flexible section is expanded lengthwise, by unfolding the grooves, with partial expansion lengthwise of the flexible section, the needle is caused to bend with respect to the axis.

The supply device is any device that can attach to the hub to contribute to either pushing matter out through the flexible section and needle, or drawing matter into the needle and flexible section. The needle, in a preferred embodiment, comprises an upper section defining a first open end and a sharpened point defining a second open end with a shaft therebetween, the needle and flexible section defining a longitudinal axis therethrough and having at least one open lumen extending from the upper section to the sharpened point.

In all embodiments of the invention, the flexible section comprises corrugated material so as allow the needle to bend while retaining the lumen open. In some embodiments, the flexible section is integrally created by corrugating the needle material at a desirable location near the proximal end of the needle. In other embodiments, the flexible section comprises a tube attachable to the hub at its proximal end and a needle at its distal end, such that the flexible member can be used with existing needles. In all embodiments, the flexible section is comprised of pliant materials, including plastics and other structurally appropriate materials.

It will be seen that in the use of the present invention the needle, attached to the flexible section, can be bent in a range from 1 degree to 90 degrees from the central axis in any direction.

A preferred embodiment of the needling system of the present invention, comprises a hollow needle having a distal end and a proximal end and a flexible section therebetween, wherein the flexible section comprises a plurality of circumferential grooves, each of the grooves having sides of unequal length. Further, the embodiment includes a hub comprising means to hold the proximal end of the hollow needle at a distal end of the hub when connected thereto, the hub being receptive to connection to a supply device at a proximal end of the hub, the hub and needle sharing a longitudinal axis therethrough when connected and in a first coaxial position. In use, the flexible section is contracted lengthwise with the sides of the grooves reentrant and overlapping when in a first straight position and when the flexible section is expanded lengthwise, by unfolding or pulling the grooves apart, the partial expansion lengthwise, of the flexible section, effects an axial bending of the needle. Locking the needle in place occurs when the grooves of the flexible section are expanded and the needle is directed to the desired angle from its axis.

The needle can be bent in a range of from 1 degree to as much as 90 degrees, in any direction, to a main axis of the needling system, to positions at least within a generally conical shape about the edge of the hub.

In a preferred embodiment of the needling system, the needle comprises an upper section defining a first opening and a sharpened point defining a second opening with a shaft therebetween, the tube defining a longitudinal axis therethrough and having at least one lumen extending from the upper section to the sharpened point. The hub contains an accordion-like flexible section with a hole or holes therethrough so as allow the needle to be secured and provide a pass through for matter. In the operation of the device then, the construction of the accordion-like segment of the hub allows the center axis of the attached needle to be positioned at a user selected angle relative to the angle of the centerline of the hub where it connects to the means for injecting or extracting matter.

In another embodiment, the accordion-like structure is constructed of the same material as the needle and its distal end is attached to proximal end of the needle. In this embodiment the proximal end of the accordion-like structure connects to yet another tubing segment where the tubing segment is also constructed of the same or similar material as the needle, the tubing segment's proximal end providing a means for connection to the distal end of the hub.

In yet another embodiment, the accordion-like structure is constructed of a material other than the material of which the needle is created, such as a polymer for instance, whereas the distal and proximal ends of the accordion-like structure are configured so as to enable attachment of the proximal end of the needle and the distal end of an additional tubing segment. As in the prior embodiment, this tubing segment's proximal end providing a means for connection/attachment to the distal end of the hub.

Each of the above-mentioned embodiments in this non-limiting series of examples may be used in different manners and to treat different ailments, or may be used under different conditions. Each embodiment will differ in manufacturing cost and complexity as well as provide varying degrees of fluid extraction or delivery precision. Depending on the treatment requirements, the user can select the appropriate embodiment.

In summary, the present invention discloses a needling system that comprises a hollow needle having a distal end and a proximal end; an accordion-like bendable segment secured thereto at its proximal end and configured to provide a pass through for matter; a hub comprising means to provide connection to the bendable segment with a distal end of the hub when connected thereto, and receptive to connection to a supply device at a proximal end of the hub, the hub and needle sharing a longitudinal axis therethrough when connected and in a first coaxial position.

A more detailed explanation of the invention is provided in the following description and claims and is illustrated in the accompanying drawings.

### BRIEF DESCRIPTION OF THE DRAWINGS

A better understanding of the present invention may be obtained with reference to the following description in conjunction with the drawings in which:

FIG. 1 is a front elevational view of a hollow needle and needling system of the prior art.

FIG. 1A is a top elevation view of the hollow needle and needling system of the prior art.

FIG. 1B is a cross sectional view of the hollow needle and needling system of FIG. 1, taken along the line B-B of FIG. 1A.

FIG. 2 is a front elevational view of a prior art hollow needle and needling system in use.

FIG. 2A is a front elevation view of prior art hollow needle and needling system in use.

FIG. 2B is a front elevation view of prior art hollow needle and needling system shown with the needle inserted.

FIG. 2C is a detailed schematic representation of the tip of a prior art hollow needle and needling system in relation to a hard to reach point of interest.

FIG. 2D is a front elevational view of prior art hollow needle and needling system.

FIG. 2E is a front elevational view of prior art hollow needle and needling system.

FIG. 3 is a perspective view of one embodiment of the hollow needle of the present invention shown with the integral bending segment and hub.

FIG. 3A is a top plan view of the hollow needle of FIG. 3.

FIG. 3B is a front plan view of the hollow needle of FIG. 3A.

FIG. 3C is a left plan view of the hollow needle of FIG. 3B

FIG. 3D is a front plan view of the hollow needle embodiment shown in FIG. 3 with the needle position in a non-co-axial position.

FIG. 3E is a left side view of the hollow needle of FIG. 3E.

FIG. 4 is a back plan view of a second embodiment of the hollow needle of the present inventions shown with the integral bending segment but without the integral hub.

FIG. 4A is a top view of the device of FIG. 4.

FIG. 4B is a back view of the device of FIG. 4 with the needle position in a non-co-axial position.

FIG. 4C is a top view of the device of FIG. 4 with the needle position in a non-co-axial position.

FIG. 5 is a perspective view of another embodiment of the bendable segment of the present invention where the segment is integrated into the hub as part of the hub and the bendable segment bent in such a manner that the needle connecting port for the hub is non-co-axial to the syringe connecting port of the hub.

FIG. 5A is a back view of the device of FIG. 5.

FIG. 5B is a top view of the device of FIG. 5A.

FIG. 5C is a back view of the device of FIG. 5 showing the integrated needle segment and the needle positioned in a non-co-axial position relative to the hub connection port.

FIG. 5D is a top view of the device of FIG. 5 showing the integrated needle segment and the needle positioned in a non-co-axial position relative to the hub connection port

FIG. 6A is a front elevation view of one embodiment of the needling system of the present invention shown in a position allowing access to the point of interest along a desired line of injection.

FIG. 6B is a front elevation view of one embodiment of the needling system of the present invention shown with the needle inserted along a line of injection and accessing the point of interest.

FIG. 6C is a detailed schematic representation of FIG. 2E showing the tip of the hollow needle of the present invention and the point of interest.

FIG. 6D is a front elevational view of one embodiment of the needling system of the present invention accessing a point of interest along a desired line of injection.

FIG. 6E is a front elevational view of one embodiment of the needling system of the present invention accessing the point of interest while avoiding internal and external obstacles.

### DETAILED DESCRIPTION OF THE ILLUSTRATIVE EMBODIMENT

While the present invention is susceptible of embodiment in various forms, there is shown in the drawings a number of presently preferred embodiments that are discussed in greater detail hereafter. It should be understood that the present disclosure is to be considered as an exemplification of the present invention, and is not intended to limit the invention to the specific embodiments illustrated. It should be further understood that the title of this section of this application (“Detailed Description of the Illustrative Embodiment”) relates to a requirement of the United States Patent Office, and should not be found to limit the subject matter disclosed herein.

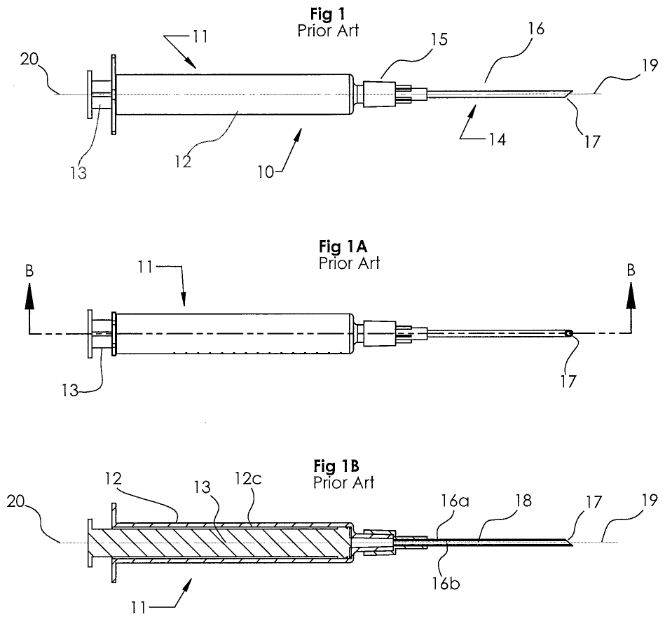

To better understand the issues in prior art needling systems, a brief review of such and the methods of using them wherein problems of accessing a point of interest are here reviewed. Referring to FIGS. 1, 1A and 1B, a needling system or cannula system 10 of the prior art is shown; it will be understood by persons having ordinary skill in the art that needling systems 10 are typically made of multiple parts: the syringe 11, consisting of two major parts, the body 12 and the plunger 13; and the needle 14 consisting of three parts, the hub 15, the shaft 16 and the bevel 17. The hub 15 is at one end of the needle and is the part that bridges between the needle and a syringe or other medical apparatus, attaching thereto. The shaft 16 is the long slender stem of the needle 14 that is beveled 17 at one end to form a point. The hollow bore of the needle is known as the lumen 18. Needle 14 is generally cylindrical and comprises an inner generally cylindrical hollow section or lumen 18 therewithin, running the entire length of the needle 14. FIG. 1B, shows a cross-sectional view, taken along the line B-B of FIG. 1A, of the needling system, including the exterior surface 16a of the needle shaft 16 and interior surface 16b of the hollow bore lumen or lumen 18 of the needle shaft 16. It will be seen that the shaft 16 of the prior art needle 14 is generally straight and when used as designed, provides access to, among other things, the interior of a body by means of puncturing the skin or other barrier, and thereby providing straight line access between an item connected to the needle and a body; and that the centerline 19 of the needle shaft 16 is parallel and typically co-linear with the center line 20 of the attached device 11, in this example a syringe 11.

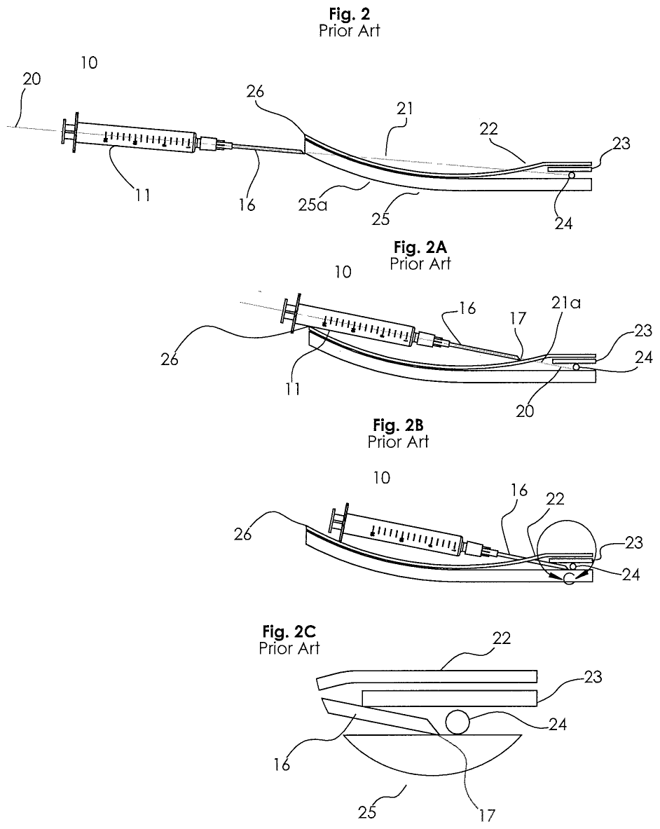

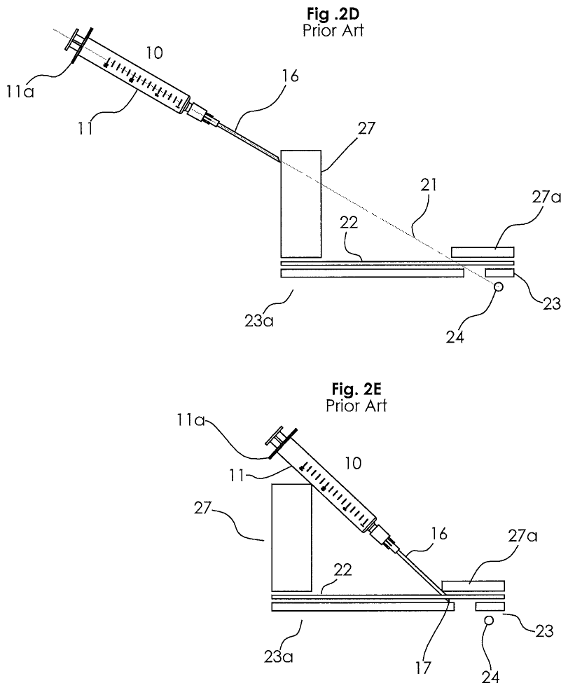

Referring now to FIG. 2, to illustrate some of the problems with the prior art needling systems, a typical prior art needling system 10 is shown along a desired injection line 21 being used to access a point of interest 24. To access point of interest 24, skin (or other membrane) 22 must be pierced and both obstructions 23 and 25 must be avoided. As shown in FIG. 2, obstruction 25 may not be linear, having some bend 25a which may occur as either a part of a single obstruction (as shown) or may occur as, for example, a joint or other obstruction (not shown). The illustrated curve 25a in the obstruction 25 causes it to block access to point of interest 24 making it impossible for the illustrated prior art needling system 10 to access point of interest 24.

Now referring to FIG. 2A, the user has positioned needling system 10 in such a manner that the syringe body 11 is placed tangentially to the obstruction's tip 26, such that needle 16 will just clear first obstruction 23. It can be seen by referring to FIG. 2B and in more detail in FIG. 2C that due to obstructions 23, 25, needle shaft 16 contacts first obstruction 23 while also having its tip 17 contact second obstruction 25. Contact occurs in such a manner that needle 16 cannot access the point of interest 24. It should also be noted that due to the limited clearance between syringe 11, skin 22 and obstruction 25, an awkward control situation is created as will be understood by persons having ordinary skill in the art. A person having ordinary skill in the art and having only the prior art before him, would suggest that a longer needle could be used here, such that the needle spanned the distance from obstruction tip 26 to target of interest 24, thus eliminating limits of the syringe 11. It will be understood, however, that where this solution is possible the solution would create a situation where the use of a longer needle can have a deleterious effect on the user's control of the procedure and more particularly the path and targeting of the needle. Further, when using a longer needle, a larger gauge needle may be required and can add to the trauma at the injection site.

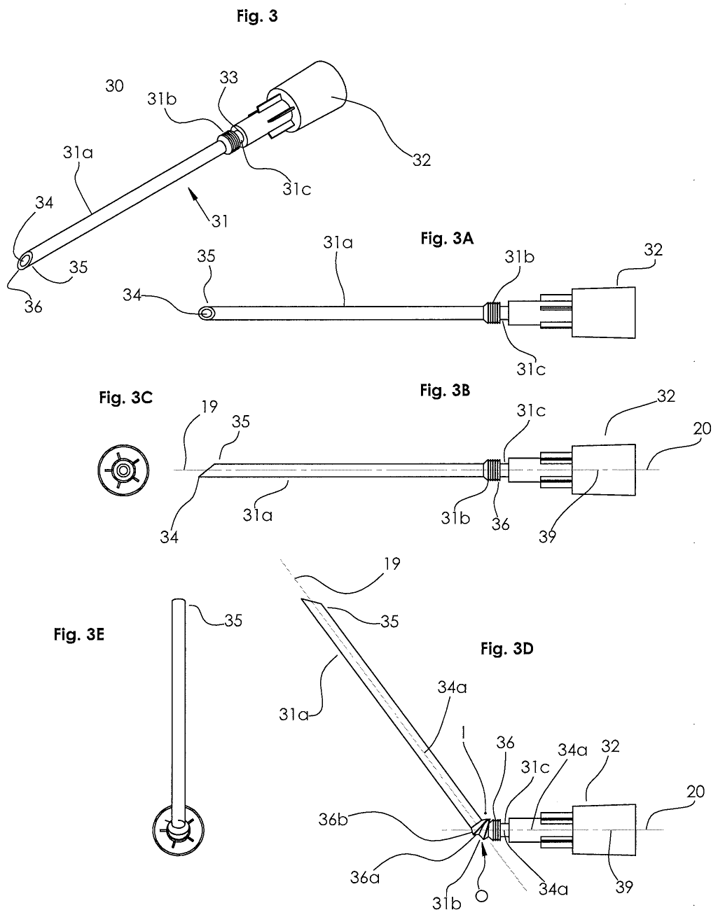

Referring now to FIG. 3, a hollow needle 30, of the present invention, is illustrated comprising a thin, tube body or needle 31, receiving a hub or syringe attachment mechanism (commonly a luer lock) 32 at end 33. Although this embodiment describes the use of a luer lock, other attachment mechanisms or hubs may be used for connection of the hollow needle 30 to a syringe or other medical device as known to persons having ordinary skill in the art, without departing from the novel scope of the present invention. As will be known to persons having ordinary skill in the art, the most common connection of medical needles is the syringe; which typically comprises a cylindrical body or tube 12 within which a plunger 13 can be moved along the longitudinal axis 20 of the syringe (see, FIGS. 1 and 1B) within a cavity 12c, designed to hold or receive liquids to inject or retract a substance from or into the syringe through the lumen 34 of needle 30 to or from a point-of-interest 24 under the skin 22. At the needle shaft distal end 35 there is a sharp tip 36 that is typically beveled, serrated, pronged, or otherwise created to facilitate penetration of the skin 22 or other tissues and or substances 23. In the present invention, the needle 31 is comprised of 3 segments of similar material properties. Segment 31a is a rigid hollow shaft with sharp tip 36 at distal end 34. The proximal end of segment 31a is connected to the distal end of a flexible hollow segment 31b. The proximal end of the flexible hollow segment 31b being connected to the distal end of another rigid hollow shaft 31c. The proximal end of 31c, end 33, in turn being connected to the distal end of hub 32. The lumen 34 of needle 31 continues into and through segments 31a, 31b, and 31c and hub 32, providing a continuity of path 34a for fluid delivery or extraction by a needling system. FIGS. 3, 3A, 3B, and 3C depict the needle shaft 31 and associated segments 31a, 31b, and 31c in an orientation such that the centerline 19 of the needle shaft 31 is aligned with the centerline 39 of hub 32.

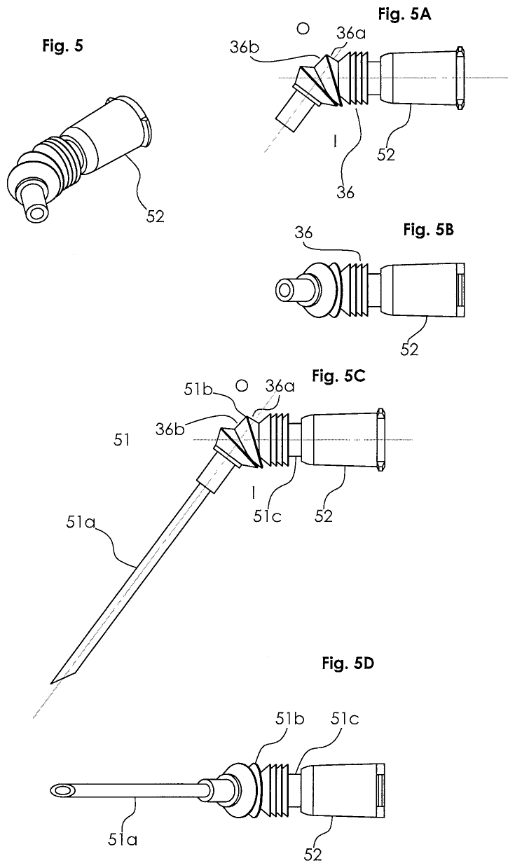

With respect to the flexible hollow segment 31b, 41b, 52b it will be understood by persons having ordinary skill in the art that it can be of any type of accordion-like element, including helical, or segmented, or a plurality of circumferential grooves each comprised of two sides of different lengths or other accordion-like configurations as are known in the art. In the preferred embodiment, as shown in FIGS. 3, 4 and 5 the flexible hollow segment 31b, 41b, 51b is comprised of a plurality of circumferential grooves 36, each of-which comprise two sides 36a and 36b. The sides 36a and 36b are of differing lengths and, as most clearly illustrated in FIGS. 5A, 5B and 5C, the side 36b is formed of a greater length than the side 36a.

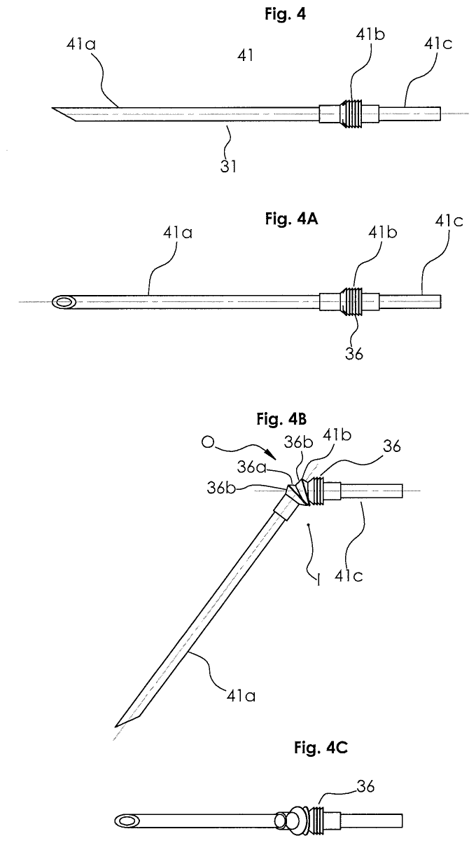

The flexible segment 31b, 41b, 51b is contracted lengthwise and this may be accomplished by applying an axially compressive force to the ends of the tube. FIGS. 3, 3A, 3B, 4 and 4A show the flexible segment 31b, 41b contracted lengthwise. This position is achieved and held primarily due to the fact that the sides 36a and 36b of grooves 36 are formed of different lengths. When an axially compressive force is applied to the flexible segment 31b, 41b, 51b the flexible segment 31b, 41b, 51b contracts lengthwise with the sides 36a and 36b being placed in a reentrant and overlapping position. As most clearly illustrated in FIGS. 3A and 4A, when the flexible segment 31b, 41b is contracted lengthwise, the sides 36a and 36b overlap thereby forming a plurality of circumferential folds. The nature of the invention illustrated herein is such that once the flexible segment 31b, 41b, 51b has been placed in the contracted position it will remain in that position unless a force is applied to unfold the grooves 36 thereby to expand lengthwise all or part of the flexible segment 31b, 41b, 51b.

FIGS. 3D, 4B and 5C show the flexible segment 31b, 41b, 51b of the present invention in a bent position. This position may be achieved by applying to the flexible segment 31b, 41b, 51 b a manual force in an appropriate manner. When this is done, it will be noted that the inner radius I of the flexible segment 31b, 41b, 51b assumes a configuration different from the outer radius O. The grooves 36 along the inner radius I remain in a folded condition with the sides 36a and 36b overlapping, while along the outer radius O the grooves 36 become unfolded, the portion thereof along the outer radius O becomes somewhat expanded due to the tendency for the grooves 36 to unfold and the sides 36a and 36b to be displaced from their overlapping position. The nature of the invention is such that once placed in a bent position, the tube will remain bent without being held by the user. This tendency of the tube to hold its bent position will operate over a relatively wide range of angles, i.e., between the straight, unbent position and a position wherein the tube is bent 180 with segments 31a, 41a, 51a parallel to and alongside segments 31c, 41c, 51c, respectively.

Now referring to FIGS. 3D and 3E, to provide access to certain points of interest that are inaccessible if the centerline 2 of syringe 11 (FIG. 1) is positioned coaxially with centerline 19 of the needle shaft 31, flexible segment 31b allows needle segment 31a to be oriented in a non-coaxial manner with hub 32, while providing a sealed fluid path between the hub 32 and the needle tip 36, as will be explained below. Due to the nature of this flexible segment 31b, the non-parallel nature of the needle 31 segment 31a relative to the syringe 11 can be adjusted by the user to provide optimal accessibility to the point of interest. It is noted that although this example is shown with a needle 31 segment 31a being a straight shaft, the flexible segment configuration shown can also be applied to non-straight needles, there by further enabling ease of accessibility to difficult to access points of interest. While in all of the above examples, the needling system 10 has included a syringe, it will be understood by persons having ordinary skill in the art that any device that can be associated with a needle and hub, including but not limited to IV devices, catheters, catheter introducers and others, without departing from the novel scope of the present invention.

Referring again to FIGS. 4 and 5, there are shown embodiments of the needle shaft segment 41a, 51a integration with flexible element 41b, 51b and rigid element 41c, 51c. In the creation of the needle with flexible segment, one method, illustrated in FIG. 3, is to form the flexible element from a material that is similar to that of needle shaft segment 31a. In turn, rigid segment 31c is of similar material to that of segments 31a and 31b. Typically, these materials would be of surgical grade stainless steel or the like used in traditional needles. Manufacture of the flexible element would be expected to be higher than the alternate embodiments shown in FIGS. 4 and 5. For this additional cost however, it is anticipated that such embodiments will provide the greatest precision with respect to applied fluid volumes. In the embodiment shown in FIG. 4 the method of manufacture for needle 41 is to fabricate the flexible segment 41b from a material such as plastic using a casting, injection molding, blow molding or other appropriate method. This results in a significant cost reduction to the previously described embodiment. Needle rigid segment 41a would be fabricated from typical needle materials such as stainless steel, as would rigid segment 41c.

Referring to FIGS. 5, 5A, 5B, 5C, and 5D, in yet another embodiment, expected to provide the lowest cost solution relative to other described embodiments, the needle rigid segment 51a attaches to the flexible segment 51b. However, unlike prior embodiments, the flexible segment 51b and rigid segment 51c are integral with hub 52 and are all 3 cast, molded, or otherwise manufactured as one integral part. In the making of the flexible segment, it will be understood that corrugations can be created in the material that comprises the flexible element, in the manner described in U.S. Pat. No. 2,094,268. It will be understood by those with ordinary skilled in the art that these examples of a means of producing a hollow shaft with a flexible segment are meant to be illustrative of ways of doing this and not meant to be a limitation on the method of creation, as there are a variety of other manufacturing and assembly methods that can be used to accomplish the desired result as known to persons having ordinary skill in the art, without departing from the novel scope of the present invention.

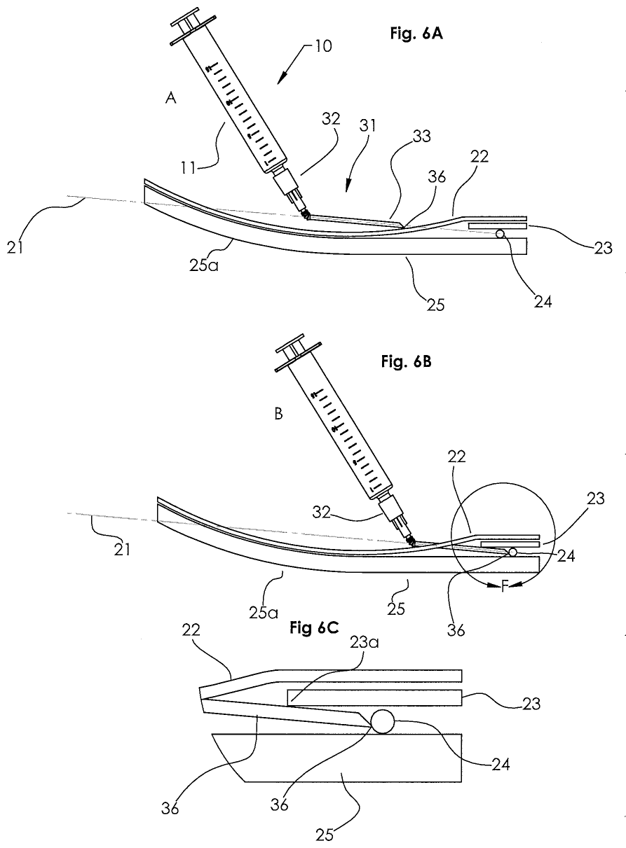

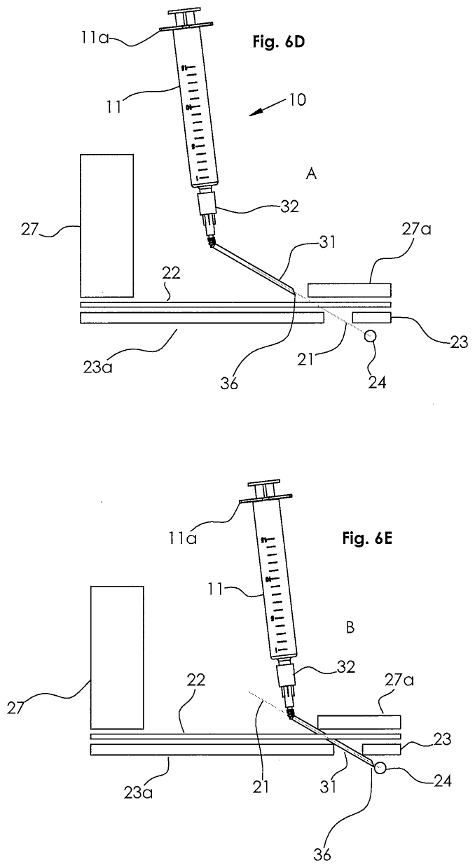

Referring now to FIG. 6A-6C, where the device of the present invention is shown, the needling system 10 of the present invention, in sharp contrast, has overcome the issues presented by the use of prior art needling systems; as will be shown and described herein. Now referring to FIGS. 6A, 6B, and 6C, the present invention includes a needling system 10 comprising a syringe 11, a hub 32 and a needle segment 31. Needle segment 31, as with the prior art, comprises a shaft 33, having a lumen 34 and a sharp tip 36. In contrast to the prior art, needle 31 can be arranged, by operation of the present invention as explained in more detail below, in a non-coaxial angle relative to hub 32 and syringe 11. Referring to FIG. 6A, it can be seen that by having needle 31 in a non-coaxial relationship with hub 32, the system 10 can be fit within the same obstruction curve 25a at which the prior art, as previously illustrated, failed. As shown in FIG. 6A, at the pre-injection position A, the needling system 10 of the present invention allows non-obstructed access to the point of interest 24 along the desired line of injection 21. Referring now to FIGS. 6B and 6C, the needling system is shown to be pushed forward, to position B, by the user, to puncture skin 22 along the desired line of insertion 21 avoiding both obstructions 23 and 25 and enabling the needle tip 36 to easily access the point of interest 24.

Referring now to FIGS. 6D and 6E, the needling system 10 of the present invention is shown, comprising a hub 32 and a needle segment 31 having sharp tip 36, where the needle 31 is positioned, by action of the present invention as will be described in greater detail below, at a non-coaxial angle relative to the hub 32 and syringe 11. It can be seen in FIG. 6D that by having needle segment 31 positioned non-coaxially with hub 32, the system 10 can be positioned at a pre-injection position A allowing non obstructed access to the point of interest 24, along the desired line of injection 21. Referring now to FIG. 6E, the needling system 10 is pushed forward, to position B, by the user to puncture skin 22 along the desired line of insertion 21 avoiding obstructions 23a, and 27a and enabling the needle 31 tip 36 to access the point of interest 24. As shown, end 11a of syringe 11 does not strike obstruction 27, gliding right by without interference in the needling process

Benefits of adjusting needle between coaxial alignments with the syringe to non-parallel position of the syringe will be obvious to persons having ordinary skill in the art. For example, one such benefit is that the aligned position can be used to draw medicine from a container then the position can then be changed to a non-aligned (non-parallel) orientation to provide ease of access to the point of interest that might otherwise be restricted from access. Further, once initial obstructions are avoided using an initial nonaligned orientation; the user can re-align or further change the desired non-alignment to meet ease of access and or ease of application needs. Due to the ease of changing alignment, such changes could occur throughout or at any point along the insertion process.

One simple example, to show the improved results of using the present invention, is in the treatment of stenosing tenosynovitis, commonly known as trigger finger. As is known, the tendons that bend the fingers glide easily with the help of the annular ligaments or pulleys. Annular ligaments are fibrous sheaths that are thick and strong as required by the repetitive use that such a system by nature necessitates. Trigger finger occurs when the pulley becomes too thick, such as through swelling due to repetitive use injury, so that the tendon cannot glide easily through the pulley; this often manifests itself in a finger that is locked in place, in a “trigger-like” shape. Traditional treatments of trigger finger include surgery, to open a section of the thick and fibrous pulley so as to allow the easier passage of the tendon therein and injection of a steroid to reduce the swelling. Oftentimes a doctor will first choose to provide an injection of a steroid between the tendon and pulley to reduce the swelling of the tendon and allow the tendon to glide through the pulley before considering surgery. In conventional treatment, a doctor will use a straight needle to inject into the finger, in a generally perpendicular motion relative to the finger, through the pulley and tendon and often to the bone. In this method, the patient will then wiggle the subject finger to allow the doctor to retract the needle, knowing to stopping the retraction when the syringe attached to the needle no longer wiggles with the finger. The lack of wiggle in the syringe then is an indication that the needle is no longer in the tendon and therefor is in the precise position, such that the doctor can inject the medicine in the space between the tendon and pulley.

This technique is problematic in that damage is done to the pulley and tendon by being pierced with the needle and the patient is subjected to pain. A better technique would be to use the same needle to pierce the finger in a generally horizontal manner and then drive the needle between the pulley and tendon. It will be understood, however, that a straight needle will not work for this, due to the angles that the needle will need to take, as a result of the compactness of the tendon and pulley system. Such methods are also exasperated when, for example, the finger is locked in the trigger position.

We have discovered that the needling system of the present invention, which as described above, can pierce the skin at one location and as a result of its configuration, be driven around the impediment to provide an appropriate dose of medicament to the exact location of the problem. Such an action can provide relief with a single treatment and not damage the pulley or tendon. The angular direction in the needle system of the present invention provides an appropriate angle to pierce the skin and drive, generally horizontally, between the tendon and pulley and there deliver the medicament where needed. Little or no damage is then done to the pulley or tendon, and the patient has less discomfort; as a result treatment is completed more quickly and is more effective.

In the treatment of trigger finger it will be understood that a finger so affected is often caused, by the condition, to be bent so that the finger itself is a barrier to an injection between the pulley and the tendon. The bent condition, as is known to persons having medical experience, typically results because the swollen or enlarged part of the tendon is trapped within the thick and fibrous pulley such that a needle cannot reach the location of the swelling, both because of the position of the finger (bent back over the palm of the hand) and because the point of interest is within the pulley.

In the use of the injection, it will be understood that because of the position of the finger, bent above the point of interest, and the pulley, which is thick and fibrous, each comprise a blockage of the space between the pulley and the tendon. For this reason, it is often the case that the space cannot be reached using the conventional treatment protocols noted above, resulting in the need for more than one treatment; causing more damage to the pulley and the tendon in an effort to provide sufficient medicament to arrive at the desired result. When the space cannot be reached, treatment may require surgery with all of the attendant issues and problems of surgery.

Although an illustrative embodiment of the invention has been shown and described, it is to be understood that various modifications and substitutions may be made by those skilled in the art without departing from the novel spirit and scope of the invention.

What is claimed is:

1. A needling system, comprising:
a hollow needle having a distal end and a proximal end, and a flexible section therebetween;
a hub comprising means to hold the proximal end of the hollow needle at a distal end of the hub when connected thereto, and receptive to connection to a supply device at a proximal end of the hub, the hub and needle sharing a longitudinal axis therethrough when connected and in a first coaxial position;
wherein the needle is pivotable, at its proximal end with a flexible section, such that the distal end of the needle can be placed at an angle relative to the longitudinal axis of the hub.

2. The needling system of claim 1, wherein the flexible section comprises a plurality of circumferential grooves, each of the grooves having sides of unequal length, the section being contracted lengthwise with the sides of the grooves reentrant and overlapping when in a first straight position.

3. The needling system of claim 2, wherein the flexible section is expandable lengthwise by unfolding the grooves, with partial expansion lengthwise of the flexible section effecting axial bending of the needle.

4. The needling system of claim 2, wherein the flexible section is formed in and as part of the hub.

5. The needling system of claim 1, wherein the supply device is any device that can attach to the hub to contribute to either pushing matter out through the flexible section and needle, or drawing matter into the needle and flexible section.

6. The needling system of claim 1, wherein the needle comprises an upper section defining a first open end and a sharpened point defining a second open end with a shaft therebetween, the tube defining a longitudinal axis therethrough and having at least one open lumen extending from the upper section to the sharpened point.

7. The needling system of claim 1, where the flexible section comprises corrugated material so as allow the needle to bend while retaining the lumen open.

8. The needling system of claim 7, where the flexible section is integrally created by corrugating the needle material at a desirable location near the proximal end of the needle.

9. The needling system of claim 1, where the flexible section comprises a tube attachable to a hub at its proximal end and a needle at its distal end

10. The needling system of claim 9, where the flexible section is comprised of a pliant material.

11. The needling system of claim 10 where the material is plastic.

12. The needling system of claim 1 where the needle can be bent in a range from 1 degree to 90 degrees from the central axis.

13. A needling system, comprising:
a hollow needle having a distal end and a proximal end and a flexible section therebetween;
wherein the flexible section comprises a plurality of circumferential grooves, each of the grooves having sides of unequal length;
a hub comprising means to hold the proximal end of the hollow needle at a distal end of the hub when connected thereto, and receptive to connection to a supply device at a proximal end of the hub, the hub and needle sharing a longitudinal axis therethrough when connected and in a first coaxial position;
the flexible section being contracted lengthwise with the sides of the grooves reentrant and overlapping when in a first straight position;
wherein the flexible section is expandable lengthwise by unfolding the grooves, with partial expansion lengthwise of the flexible section effecting axial bending of the needle.

14. The needling system of claim 13, wherein locking the needle in place occurs when the grooves of the flexible section are expanded and the needle is directed to the desired angle from its axis.

15. The needling system of claim 13, wherein the supply device is any device that can attach to the hub to contribute to either pushing matter out through the hub flexible section and needle, or drawing matter into the needle and flexible section.

16. The needling system of claim 13, where the flexible section comprises a corrugated material having a hole therethrough so as allow the needle to bend away from the central axis of the needling system while retaining the lumen therethrough open.

17. The needling system of claim 16, where the flexible section is integrally created by corrugating the needle material at a desirable location near the proximal end of the needle.

18. The needling system of claim 13, wherein the flexible section comprises a tube attachable to a hub at its proximal end and a needle at its distal end.

19. The needling system of claim 18, where the flexible section is comprised of a pliant material.

20. The needling system of claim 19 wherein the wherein the material is plastic.

21. The needling system of claim 13 wherein the needle can be bent from the central axis of the needling system in a range from 1 degree to as much as 90 degrees to a main axis of the needling system, to positions at least within a generally conical shape about the edge of the hub.
